(19) United States
(12) Patent Application Publication
(10) Pub. No.: US 2005/0080341 A1
(43) Pub. Date: Apr. 14, 2005

(54) Seat frame for use with an extracorporeal high intensity focus ultrasonic wave therapeutic apparatus

(76) Inventors: Shenxu He, Beijing (CN); Jinsheng Yu, Beijing (CN); Jiang Lan, Beijing (CN); Liulin Xiong, Beijing (CN); Xiadong Wu, Beijing (CN)

Correspondence Address: DORSEY & WHITNEY LLP;INTELLECTUAL PROPERTY DEPARTMENT, 4 EMBARCADERO CENTER, SUITE 3400, SAN FRANCISCO, CA, 94111, US

(21) Appl. No.: 10/494,789

(22) PCT Filed: Mar. 15, 2002
(86) PCT No.: PCT/CN02/00166

(30) Foreign Application Priority Data
Nov. 5, 2001 (CN) 01134486.5

(51) Int. Cl. A61B 8/00; A61B 8/12; A61B 8/14

(52) U.S. Cl. 600/437; 600/439

(57) ABSTRACT

the present invention provides a seat frame for use with an extracorporeal HIFU therapeutic apparatus, in which the HIFU therapeutic apparatus includes a HIFU source for providing HIFU; a carrying device for patient and a displacement system for causing the spatial movement of the carrying device for patient with respect to the HIFU source, a containing means for receiving the transmission medium in front of an emiting surface of the HIFU source, said containing means for the transmission medium is an open-type water tank, wherein said seat frame includes a cushion which may be submerged in said open-type water tank, in said cushion is provided an arrangement through which the power ultrasonic wave may pass, said seat frame further includes a cushion supporting arrangement for supporting said cushion on said carrying device for patient.

### FIELD OF THE INVENTION

The present invention relates to a means for use with a medical apparatus, especially to a seat frame which is mounted for a lower-mounted ultrasonic wave source of a high intensity focus ultrasonic wave (HIFU) therapeutic apparatus for treating the pathological tissues in the body parts like the pelvic cavity.

### BACKGROUND OF THE DISCLOSURE

At present, the existing extracorporeal HIFU therapeutic apparatus generally includes the following means: a HIFU source and a driving circuit thereof for providing the HIFU; a locating system for searching the object to be treated and moving it to the focal point of the ultrasound transducer, which includes a medical imaging system (such as the B-Mode Ultrasonoscope), a carrying device for carrying the patient such as a bed surface, and a displacement system for causing the spatial movement of the carrying device with respect to the ultrasonic wave source; a high intensity ultrasonic wave (HIU) transmitter and a processing system for transmission medium. Since the ultrasonic wave adapted to the HIFU must be transmitted into the patient body through a special transmission medium (such as deaerated water), a containing means for receiving the transmission medium (such as a water tank etc.) in front of a emitting surface of a HIFU source and means for filling, discharging and processing the transmission medium are necessary.

The high intensity ultrasonic wave (HIU) for therapy can not pass through some substance such as bones and air. All the existing extracorporeal HIFU therapeutic apparatus use the bed surface as a carrying device for patient and the patient is in a pronation pose when being treated. However, it is difficult for the HIU to reach an object to be treated from the front or the back of the patient due to the influence of the pelvic cavity on the diseased tissue in the rectum, prostate and unerine neck and so on, so that the diseased tissues in those parts can not be treated.

### SUMMARY OF THE INVENTION

The object of the present invention is to overcome the above-mentioned disadvantages by providing a seat frame for use with an extracorporeal HIFU therapeutic apparatus, by which the patient can be in a sitting pose when being treated so that the the treatment range of the HIFU can be extended.

In order to fulfill the above object, the present invention provides a seat frame for use with an extracorporeal HIFU therapeutic apparatus, wherein the HIFU therapeutic apparatus includes a HIFU source for providing HIFU; a carrying device for carrying the patient and a displacement system for causing the spatial movement of the carrying device for patient with respect to the HIFU source. A containing means for receiving the transmission medium is provided in front of an emitting surface of the HIFU source, said containing means for the transmission medium is an open-type water tank, and said seat frame includes a cushion which may be submerged in said open-type water tank. In the center of said cushion is provided an arrangement through which the power ultrasonic wave may passes, and the seat frame further includes a cushion supporting arrangement for supporting said cushion on said carrying device.

The seat frame of the present invention can further includes the following features: said arrangement through which the power ultrasonic wave can pass is a center hole or a latex membrane, and said cushion is made of a softer and resilient material such as a foam plastic.

Said cushion supporting arrangement includes a frame, said cushion is placed on a base plate made of a rigid material, the shape of said base plate corresponds to that of said cushion, and a hole corresponding to said arrangement of the cushion through which the ultrasonic wave can pass is formed in said base plate, said cushion supporting arrangemet further includes bearing plates provided on both sides of said cushion respectively, said bearing plate has one end connected to the base plate for cushion and the other end connected to said frame, so that the cushion supporting arrangement supports said cushion. One end of said bearing plate is connected to said base plate by a fastener, and the other end can also be connected to said frame by a fasterner.

Further, said cushion supporting arrangement includes a backrest and an armrest connected to said frame.

In use, said seat frame is mounted above said open-type water tank which is above the lower-mounted HIFU source. When said water tank is full of deaerated water, the water level will exceed the cushion of said seat frame, that is, said cushion will be submerged in the water. The patient sits on the seat frame when being treated and the perineum skin sufficiently contact the deaerated water. The ultrasonic wave will pass through said arrangement through which the power ultrasonic wave may pass and through the perineum, and then enter into the body so as to reach the parts like the rectum, prostate and unerine neck and so on so that the the pathological tissues in the body parts like pelvic cavity can be treated externally. Thus, the HIFU therapeutic apparatus allows the patient being treated in a sitting pose so as to treat the rectal cancer in situ, Mile's partial relapse of rectal cancer, prostatic cancer, cervical carcinoma, carcinoma of vagina and prostatic hyperplasia and so on, which extends the treatment range of the HIFU.

### BRIEF DESCRIPTION OF DRAWINGS

The present invention will be described in detail by the preferred embodiments with reference to the following drawings, wherein,

FIG. 1 and 2 are two perspective views of one embodiment of the seat frame according to the presnt invention seen in different directions;

FIG. 3 is a schematic view of the embodiment of the seat frame shown in FIG. 1 when being mounted on a HIFU therapeutic apparatus;

FIG. 4 is a partial sectional view of the embodiment of the seat frame shown in FIG. 1.

### THE BEST MODE OF THE EMBODIMENTS

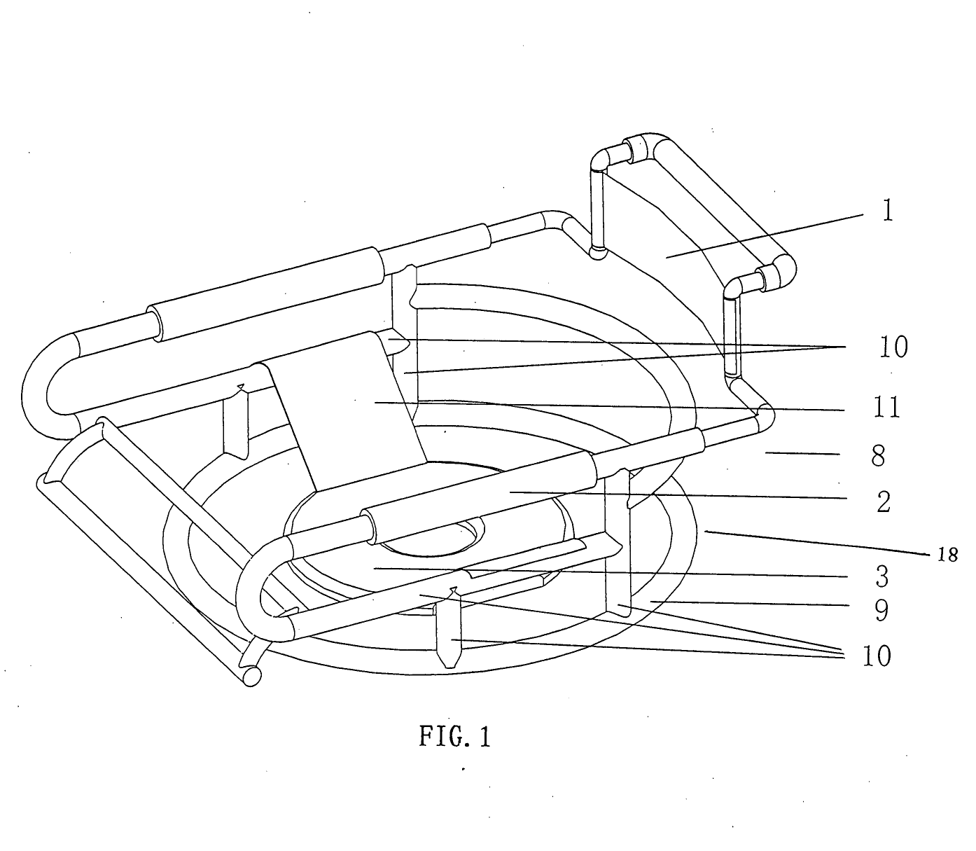

FIG. 1 shows a seat frame 8 for use with an extracorporeal HIFU therapeutic apparatus. The HIFU therapeutic apparatus includes a HIFU source 6 for providing HWFU; a carrying device 7 for carrying the patient and a displacement system (not shown) for causing the spatial movement of the carrying device 7 with respect to the HIFU source 6, a containing means for receiving the transmission medium in front of an emitting surface of the HIFU source 6 (In this embodiment, said containing means for the transmission medium is an open-type water tank 5.), wherein said seat frame includes a cushion 3 which may be submerged in said open-type water tank 5, in the center of said cushion 3 is provided an arrangement through which the power ultrasonic wave may pass, said seat frame further includes a cushion supporting arrangement 18 for supporting said cushion 3 on said carrying device 7.

In the embodiment, said arrangement through which the power ultrasonic wave is a center hole 4, or may be an arrangement like a latex membrane through which the power ultrasonic wave can pass.

Said cushion 3 is made of a softer and resilient material such as foam plastic.

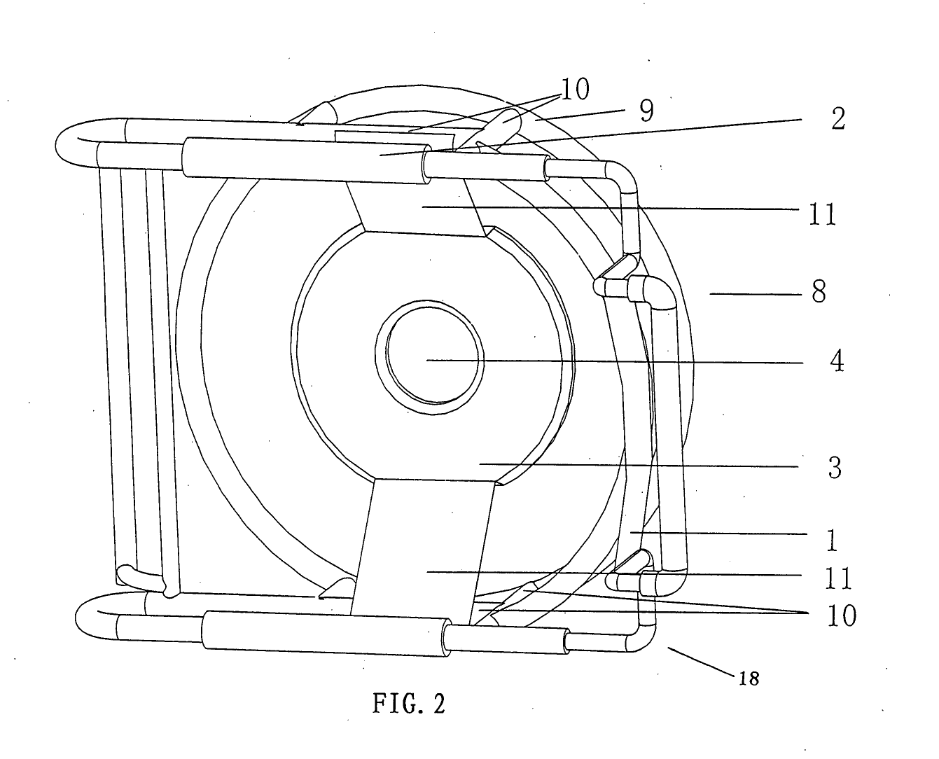

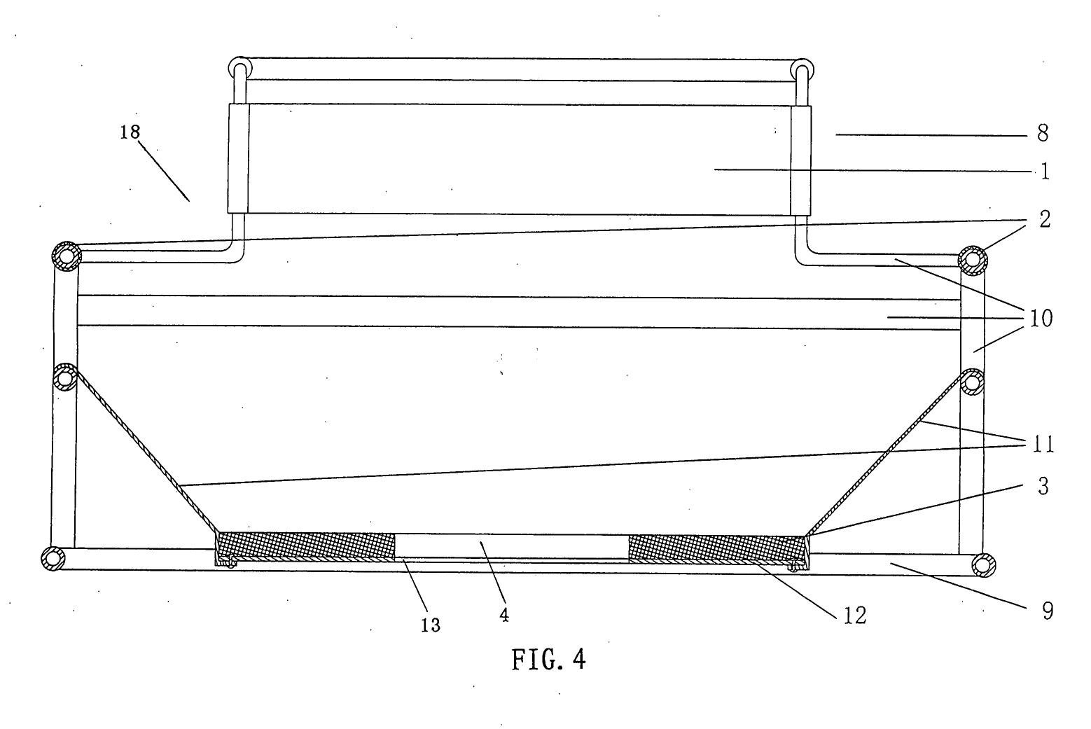

As shown in FIGS. 1-4, said cushion supporting arrangemet 18 includes a frame 10, said cushion 3 is placed on a base plate 12 made of an rigid material, the shape of said base plate 12 corresponds to that of said cushion 3, and a hole 13 corresponding to said arrangement of the cushion 3 through which the ultrasonic wave can pass (in this embodimet, such arrangement is the center hole 4) is formed in said base plate 12, said cushion supporting arrangemet 18 further includes bearing plates 11 provided on both sides of said cushion 3 respectively. Said bearing plate 11 has one end connected to the base plate 12 for cushion and the other end connected to said frame 10, so that the base plate 12 supports said cushion on said cushion supporting arrangemet 18.

One end of said bearing plate 1 1 is connected to said base plate 12 by a fastener or welding. Alternatively, the bearing plate 11 is formed integrally with the base plate. The other end of said bearing plate 11 is conneted to said frame 10 by a fasterner or welding, too.

Further, said cushion supporting arrangemet 18 includes a base 9 for supporting the whole arrangements of the seat frame, said frame 10 is fixedly attached to said base 9, wherein the base 9 is a rigid annular ring.

In addition, said cushion supporting arrangemet 18 includes a backrest 1 and an armrest 2 connected to said frame 10.

Said seat frame 8 and said carrying device 7 for patient share a set of displacement system.

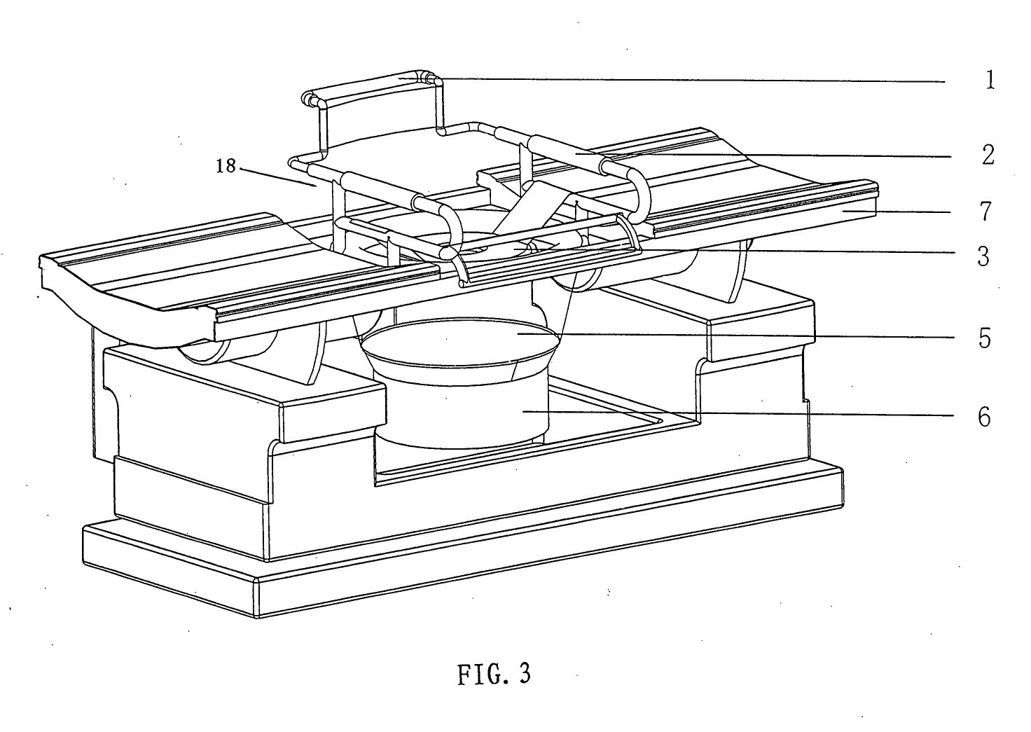

In use, said seat frame 8 is mounted above said open-type water tank 5 which is above the lower-mounted HIFU source 6, said cushion supporting arrangemet 18 of said seat frame 8 is placed on said carrying device 7 for patient, said cushion 3 is supported on the said carrying device 7 for patient, so that said seat frame 8 and said carrying device 7 for patient share a set of displacement system, which can make said seat frame move with respect to the ultrasonic wave source 6 so as to adjust it to a desirable therapy poistion, as shown in the FIG. 3. When said water tank 5 is full of deaerated water, the water level will exceed said cushion 3 of said seat frame 8, that is, said cushion 3 is submerged in the open-type water tank 5. When being treated, the patient sits on the cushion 3 in sitting pose and make the pudendal skin sufficiently contact the deaerated water, the ultrasonic wave will pass through said center hole 4 of said cushion 3 and the perineum, and then enter into the body so as to reach the parts like the rectum, prostate and unerine neck and so on so that the the pathological tissues in the body parts like pelvic cavity can be treated by HIFU. Thus, the HIFU therapeutic apparatus allows the patient being treated in a sitting pose so as to treat the rectal cancer in situ, Mile's partial relapse of rectal cancer, prostatic cancer, cervical carcinoma, carcinoma of vagina and prostatic hyperplasia and so on, which extends the treatment range of the HIFU.

FIG. 3 shows the seat frame 8 mounted on a HIFU therapeutic apparatus.

Specifically, on the basis of the HIFU therapeutic apparatus which uses a bed surface, an upper-mounted ultrasonic wave source and a lower-mounted ultrasonic wave source, the carrying device for patient can be made to be detachable. If it is necessary for the patient to be treated in a sitting pose, the middle portion of the carrying device is detached so as to expose the water tank 5 of the lower-mounted ultrasonic wave source 6 and the seat frame 8 of the present invention is mounted. The seat frame 8 and said carrying device 7 for patient share a set of displacement system so that the change between the two different therapy poses is very convenient.

In addition, for the HIFU therapeutic apparatus with a lower-mounted ultrasonic wave source 6 and an open-type water tank 5, if it is necessary for the patient to be treated in a sitting pose, what to do is to place the seat frame on the carrying device 7 for patient and to place the cushion 3 in the water tank 5.

In summary, with the seat frame, the HIFU therapeutic apparatus can treat the rectal cancer in situ, Mile's partial relapse of rectal cancer, prostatic cancer, cervical carcinoma, carcinoma of vagina and prostatic hyperplasia and so on, which extends the treatment range of the HIFU. With the present inveion, the patient can be in a sitting pose when being treated so that the the treatment range of the HIFU can be extended.

1. A seat frame for use with an extracorporeal HIFU therapeutic apparatus, in which the HIFU therapeutic apparatus includes a HIFU source for providing HIFU; a carrying device for carrying the patient and a displacement system for causing the spatial movement of the carrying device for patient with respect to the HIFU source, container for receiving the transmission medium in front of an emitting surface of the HIFU source, in which said container for the transmission medium is an open-type water tank, wherein said seat frame includes a cushion which may be submerged in said open-type water tank, in the center of said cushion is provided an arrangement through which the power ultrasonic wave may pass, said seat frame further including a cushion supporting arrangement (18) for supporting said cushion on said carrying device for said patient.

2. A seat frame for use with an extracorporeal HIFU therapeutic apparatus according to claim 1, wherein said arrangement through which the power ultrasonic wave can pass is a center hole.

3. A seat frame for use with an extracorporeal HIFU therapeutic apparatus according to claim 1, wherein said arrangement through which the power ultrasonic wave can pass is a latex membrane.

4. A seat frame for use with an extracorporeal HIFU therapeutic apparatus according to claim 1, wherein said cushion is made of a soft and resilient material.

5. A seat frame for use with an extracorporeal HIFU therapeutic apparatus according to claim 4, wherein said cushion is made of a foam plastic.

6. A seat frame for use with an extracorporeal HIFU therapeutic apparatus according to claim 1, wherein said cushion supporting arrangement includes a frame, said cushion is placed on a base plate made of a rigid material, the shape of said base plate corresponds to that of said cushion, and a hole corresponding to said arrangement of the cushion through which the ultrasonic wave can pass is formed in said cushion base plate, said cushion supporting arrangement further includes bearing plates provided on both sides of said cushion respectively, said bearing plate has one end connected to the cushion base plate and the other end connected to said frame, so that the base plate supports said cushion on said cushion supporting arrangement.

7. A seat frame for use with an extracorporeal HIFU therapeutic apparatus according to claim 6, wherein one end of said bearing plate connected to said base plate by a fastener and the other of said bearing plate is connected to said frame by a fastener.

8. A seat frame for use with an extracorporeal HIFU therapeutic apparatus according to claim 6, wherein said cushion supporting arrangement includes a base for supporting the whole arrangements of the seat frame, said frame is fixedly attached to said base.

9. A seat frame for use with an extracorporeal HIFU therapeutic apparatus according to claim 8, wherein the base is a rigid annular ring.

10. A seat frame for use with an extracorporeal HIFU therapeutic apparatus according to claim 6, wherein said cushion supporting arrangement includes a backrest and an armrest connected to said frame.
